(19) United States
(12) Patent Application Publication
(10) Pub. No.: US 2019/0336789 A1
(43) Pub. Date: Nov. 7, 2019

(54) Methods of Controlling Lighting Systems for Light Related Health and Systems Incorporating the same

(71) Applicants: Richard C. Garner, Arlington, MA (US); Christopher P. Scarlata, Billerica, MA (US)

(72) Inventors: Richard C. Garner, Arlington, MA (US); Christopher P. Scarlata, Billerica, MA (US)

(73) Assignee: OSRAM GmbH, Munich (DE)

(21) Appl. No.: 15/963,208

(22) Filed: Apr. 26, 2018

(51) Int. Cl. A61N 5/06

(52) U.S. Cl. CPC A61N 5/0618; A61M 2021/0044; A61N 5/0621

(57) ABSTRACT

Methods and systems are described that enable the simultaneous control of lux, correlated color temperature (CCT), and circadian light (CL) emitted by a lighting system. Aspects also include methods for the adjusting the lux, CCT, and CL with respect to the needs and/or desires of a user, including to provide optimal lighting for light related health. Aspects of the present disclosure also include user interfaces for the simultaneous knowledge and control of lux, CCT, and CL.

### FIELD OF THE DISCLOSURE

The present disclosure generally relates to the field of methods of controlling lighting systems. In particular, the present disclosure is directed to methods of controlling lighting systems for light related health and lighting systems with light related health controls.

### BACKGROUND

An organism's circadian rhythm is heavily influenced by the characteristics of the lighting the organism is exposed to throughout the day. In humans, melatonin, a hormone secreted by the pineal gland in the hypothalamus, regulates the circadian rhythm, and light exposure influences the amount of melatonin the pineal gland secretes. Light exposure, therefore, influences the body's regulation of sleep patterns and other biological functions. And the unnatural suppression of melatonin, which can occur from, for example, missing sleep or changing time zones, can contribute to sleep disorders, disturb the circadian rhythm, and may also contribute to adverse health conditions such as hypertension, heart disease, diabetes, and cancer.

Blue light, and the blue light component of polychromatic light, have been shown to suppress the secretion of melatonin. Moreover, melatonin suppression has been shown to be wavelength dependent, and peak at wavelengths between about 420 nm and about 480 nm. As such, an individual's circadian rhythm can be adversely impacted if he or she is exposed to an excess amount of blue light over a long duration of time. Conversely, an individual's health may be improved by controlling the melatonin-suppression or promotion characteristics of light he or she is exposed to.

### SUMMARY OF THE DISCLOSURE

In one implementation, the present disclosure is directed to a method of controlling a lighting device having at least two differently colored channels of solid state light sources. The method includes independently controlling an output of the at least two channels to simultaneously control a lux, correlated color temperature (CCT), and circadian light (CL) of light emitted by the lighting device.

In some embodiments, the at least two channels include a blue channel including a plurality of blue solid state light sources and a white channel including a plurality of white solid state light sources, the method further includes controlling the CL of the light emitted by the lighting device predominately with the blue channel and controlling the lux of the light emitted by the lighting device predominately with the white channel. In some embodiments, the at least two channels include a first white channel including a plurality of white solid state light sources having a first CCT and a second white channel including a plurality of white solid state light sources having a second CCT, the first and second CCTs being different. In some embodiments, the at least two channels further include a blue channel including a plurality of blue solid state light sources.

In some embodiments, the method further includes receiving target values for the lux, CCT, and CL of light emitted by the lighting device, receiving lighting information that includes the lux, CCT, and CL of light output by the lighting device as a function of a driving current or voltage for each of the at least two channels, and determining, with the lighting information, a driving current or voltage for each of the at least two channels to emit light from the lighting device having the target values of lux, CCT, and CL. In some embodiments, the method further includes receiving a calibration database that includes a lux table, a CCT table, and a CL table that define the lux, CCT, and CL, respectively, of light output by the lighting device as a function of driving current or voltage for the at least two channels, and determining an output for each of the at least two channels according to the calibration database. In some embodiments, the method further includes receiving a target lux and CCT, determining a range of CL values that is achievable at the target lux and CCT, determining whether CL should be minimized or maximized, determining a target CL within the range of achievable CL values, and determining an output of the at least two channels to emit a combined light having the target lux, CCT, and CL. In some embodiments, the method further includes receiving at target value for at least one of the lux, CCT, and CL of light output by the lighting device, determining a range of achievable values for the other ones of the lux, CCT, and CL of the light output, determining a target value within the range of achievable values for each of the other ones of lux, CCT, and CL, and determining a driving current or voltage for each of the at least two channels to emit a combined light output having the target lux, CCT, and CL. In some embodiments, the method further includes receiving a target lux and CCT, receiving a CL instruction, determining a target CL of the light output by the lighting device according to the target lux and CCT and the CL instruction, determining a driving current for each of the at least two channels to emit a combined light having the target lux, CCT, and CL, and sending a control signal to the lighting device to emit light having the target lux, CCT, and CL.

In some embodiments, the method further includes providing a user interface (UI) that includes control features for simultaneously controlling the CL and lux of the light output by the lighting device. In some embodiments, the method further includes providing a user interface (UI) that includes control features for simultaneously controlling the CL, lux, and CCT of the light output by the lighting device.

In another implementation, the present disclosure is directed to a lighting system. The lighting system includes a lighting device including at least two differently colored channels of solid state light sources; and a processor coupled to the lighting device and configured to independently control an output of the at least two channels to simultaneously control a lux, correlated color temperature (CCT), and circadian light (CL) of light emitted by the lighting device.

In some embodiments, the at least two channels include a blue channel including a plurality of blue solid state light sources and a white channel including a plurality of white solid state light sources, and the processor is further configured to control the CL of the light emitted by the lighting device predominately with the blue channel and control the lux of the light emitted by the lighting device predominately with the white channel. In some embodiments, the at least two channels include a first white channel including a plurality of white solid state light sources having a first CCT and a second white channel including a plurality of white solid state light sources having a second CCT, the first and second CCTs being different. In some embodiments, the system further includes a non-transitory computer readable medium containing lighting information that includes the lux, CCT, and CL of light output by the lighting device as a function of a driving current or voltage for each of the at least two channels, and the processor is further configured to receive target values for the lux, CCT, and CL of light emitted by the lighting device, and determine, with the lighting information, a driving current or voltage or each of the at least two channels to emit light with the lighting device having the target values of lux, CCT, and CL. In some embodiments, the system further includes a non-transitory computer readable medium containing a calibration database that includes a lux table, a CCT table, and a CL table that define the lux, CCT, and CL, respectively, of light output by the lighting device as a function of a driving current or voltage for the at least two channels, and the processor is further configured to determine an output for each of the at least two channels according to the calibration database.

In some embodiments, the processor is further configured to receive a target lux and CCT, determine a range of CL values that is achievable at the target lux and CCT, determine whether CL should be minimized or maximized, determine a target CL within the range of achievable CL values, and determine an output of the at least two channels to emit a combined light having the target lux, CCT, and CL. In some embodiments, the processor is further configured to receive at target value for at least one of the lux, CCT, and CL of light output by the lighting device, determine a range of achievable values for the other ones of the lux, CCT, and CL of the light output, determine a target value within the range of achievable values for each of the other ones of lux, CCT, and CL, and determine a driving current or voltage for each of the at least two channels to emit a combined light output having the target lux, CCT, and CL. In some embodiments, the processor is further configured to receive a target lux and CCT, receiving a CL instruction, determine a target CL of the light output by the lighting device according to the target lux and CCT and the CL instruction, determine a driving current for each of the at least two channels to emit a combined light having the target lux, CCT, and target CL, and send a control signal to the lighting device to emit light having the target lux, CCT, and CL. In some embodiments, the processor is further configured to provide a user interface (UI) that includes control features for simultaneously controlling the CL, lux, and CCT of the light output by the lighting device.

### BRIEF DESCRIPTION OF THE DRAWINGS

For the purpose of illustrating the disclosure, the drawings show aspects of one or more embodiments of the disclosure. However, it should be understood that the present disclosure is not limited to the precise arrangements and instrumentalities shown in the drawings, in which:

FIG. 1 is a block diagram of an example lighting system with light related health controls;

FIG. 2A is a functional block diagram of an example lighting device;

FIG. 2B is a functional block diagram of another example of a lighting device;

FIG. 3 is a graph of spectral emission curves for the example lighting device of FIG. 2B;

FIG. 4 is a CIE chromaticity diagram for the lighting channels of the example lighting device of FIG. 2B;

FIG. 5A is a contour plot of a circadian light metric CL_{A} for light emitted by the lighting device of FIG. 2B for various combinations of the lux of the white channel and ratios of intensity of the blue channel to the white channel;

FIG. 5B is a contour plot of a circadian light metric circadian stimulus (CS) for light emitted by the lighting device of FIG. 2B as a function of the lux of the white channel and ratios of intensity of the blue channel to the white channel;

FIG. 5C is a contour plot of the CCT of light emitted by the lighting device of FIG. 2B as a function of the lux of the white channel and ratios of intensity of the blue channel to the white channel;

FIG. 5D is a contour plot of the total lux of the light emitted by the lighting device of FIG. 2B as a function of the lux of the white channel and ratios of intensity of the blue channel to the white channel;

FIG. 5E is a contour plot of the lux of light emitted by the blue channel of the lighting device of FIG. 2B as a function of the lux of the white channel and ratios of intensity of the blue channel to the white channel;

FIG. 6 is a contour plot of CS for light emitted by the lighting device of FIG. 2B as a function of the lux of the white channel and the CCT of light emitted by the lighting device;

FIG. 7 is a contour plot of CS for light emitted by the lighting device of FIG. 2B as a function of the lux of the white and blue channels;

FIG. 8 is a functional block diagram of tables of data that may be included in the calibration database of the lighting system of FIG. 1;

FIG. 9 is an example user interface (UI) for controlling a lighting system for light related health (LRH);

FIG. 10 is another example of a UI for controlling a lighting system for LRH;

FIG. 11 is a flowchart of an example method of controlling a lighting device;

FIG. 12 is a graph of four spectral responsivity curves of the human circadian system to light;

FIG. 13 is a graph of CS for a blue LED and four blackbodies of different temperatures; and

FIG. 14 shows a diagrammatic representation of one embodiment of a computing device that may be used for implementing lighting control methods of the present disclosure.

### DETAILED DESCRIPTION

The present disclosure includes lighting control systems and methods, adaptable to a variety of lighting systems, that enable the simultaneous control and knowledge of lux, correlated color temperature (CCT), and circadian light (CL) emitted by the lighting system. The present disclosure also includes algorithms for the optimal setting of these three parameters with respect to needs and/or desires of a user. In some examples, the light related health (LRH) needs of a user may, for example, be determined by a personal wearable device that monitors the light experienced by the user during the course of the day.

The term circadian light, or CL, as used herein refers to a metric which gives an indication of the strength of a light's effect on the human circadian system (HCS). In some examples, the term CL refers to the tendency of a light source to suppress the secretion of melatonin in an organism. As discussed more below, control systems of the present disclosure can employ any of a variety of particular CL metrics, with one example metric provided by way of example.

In some examples, the three parameters, lux, CCT, and CL, are not all independent of each other, and optimal settings for a lighting system involve certain imposed constraints. For example, a user may want to increase the CL, which may involve increasing the “blue-ness” of the light, but still maintain a relatively warm light (more red-ness) and a certain lux (e.g., dim) level. Methods of the present disclosure include algorithms for finding a maximum or minimum one of the three control parameters, e.g., a maximum or minimum circadian light, subject to a maximum or minimum CCT and lux value.

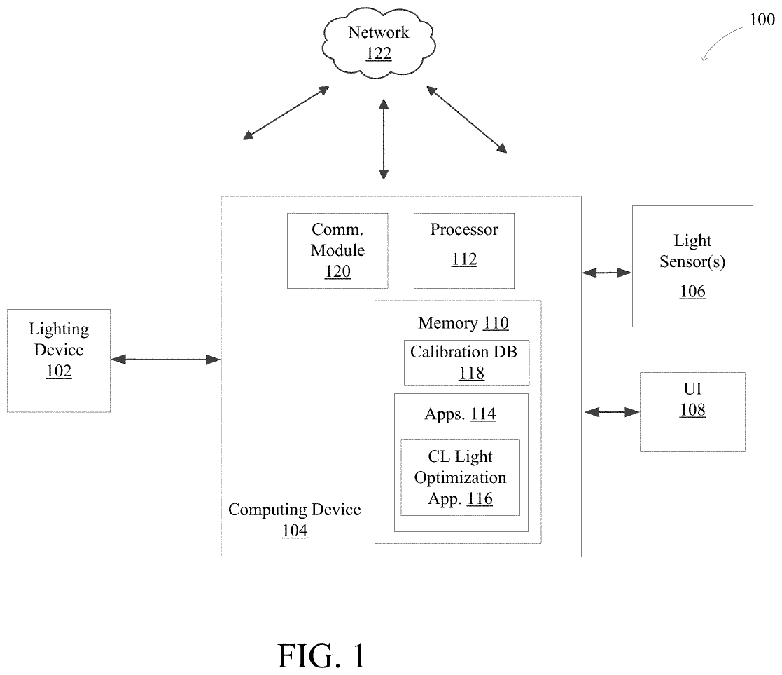

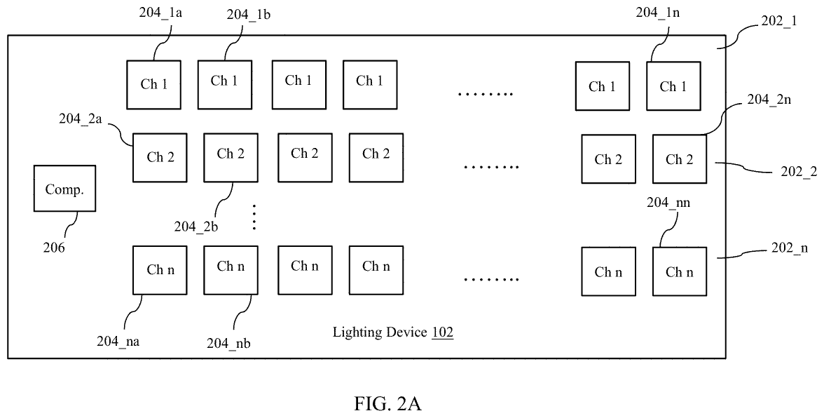

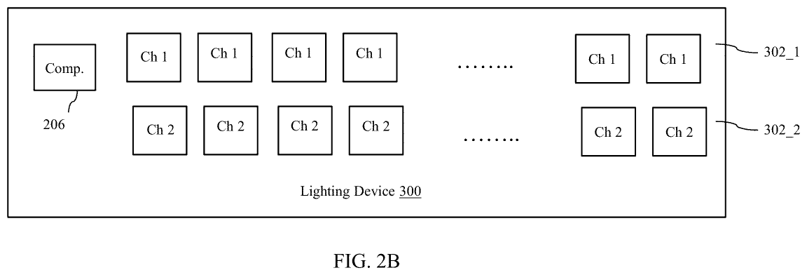

FIG. 1 is a block diagram of an example lighting system 100 for controlling one or more lighting devices 102. As described more below, each lighting device includes two or more differently-colored and independently-controllable channels (202_1-n (FIG. 2) of solid state light sources. Lighting devices 102 can be designed and configured for any of a variety of applications, for example, lamps positioned on a ceiling or elsewhere for illuminating a space, such as a place where an individual works or lives (e.g., office, laboratory, bedroom, living room, family room). Lighting devices 102 may also be designed for use in other spaces such as airplanes, buses, and trains (e.g., personal lights above seats), showers and/or baths, embedded in headwear such as glasses or helmets that a user may use during the course of normal activities (biking, motorcycling), or helmets specifically designed to be worn for light related health.

Lighting system 100 also includes a computing device 104 operably connected to lighting device 102. The computing device 104 may be a smart phone, tablet, laptop, wearable device, desktop, server, remote control, or any other electronic device capable of communicating with and controlling light sources. Computing device 104 is configured to simultaneously and independently control the CCT, lux, and CL output of lighting device(s) 102 that, as described more below, can allow for the independent control of each of CCT, lux, and CL within certain constraints. Such independent control can allow for adjustment of CL over a range of possible values while maintaining the light output of lighting device 102 at a desired CCT and lux. Enabling the control of CL independent of CCT and lux allows for CL adjustments, for example, for the purpose of LRH, while maintaining a desired or appropriate CCT and lux for a given activity.

Lighting system 100 may also include one or more light sensors 106 and a user interface (UI) 108 for providing target CCT, lux, and CL values to computing device 104. Light sensors 106 can include, for example, light sensors positioned at one or more fixed locations within a space and may also include light sensors housed in a personal wearable device that monitors the light experienced by a user during the course of the day. Light sensors 106 positioned at one or more locations within a space can be used by computing device 104 for closed loop feedback, providing actual values of CCT, lux, and/or CL within a space for comparison to target values. Any of a variety of personal wearable devices that monitor the light experienced by a user may be used. As is known in the art, such wearable devices can determine a target CL value for a given user based on, for example, the time of day, the light conditions the user has experienced during the day, and any user-specific settings. UI 108 can allow for user-specified control of lighting conditions within a space and may have any form known in the art of lighting UI design, including any combination of control features such as switches, dimmer slides, etc., implemented with physical hardware and/or a software-generated UI displayed on a display screen of a wall mounted and/or portable computing device. UI 108 may allow for direct control of one or more of lux, CCT, and CL, and/or may include user-selectable operating modes for specified tasks or times of day that include defined values of one or more of lux, CCT, and CL. Other control functions may include beam direction, beam angle, beam distribution, and/or beam diameter thereby allowing for customizing a spot size, position, and/or distribution of light in a given space or on a given surface of incidence.

In accordance with some embodiments, computing device 104 may include a memory 110. Memory 110 can be of any suitable type (e.g., RAM and/or ROM, or other suitable memory) and size, and in some cases may be implemented with volatile memory, non-volatile memory, or a combination thereof. Memory 110 may be utilized, for example, for processor workspace and/or to store media, programs, applications, content, etc., on a temporary or permanent basis. Also, memory 110 can include one or more modules stored therein that can be accessed and executed, for example, by processor(s) 112.

Memory 110 may include one or more applications 114 stored therein. Applications 114 may include a CL light optimization application 116 for determining an optimized CL light output value. For example, computing device 104 may receive or otherwise determine target values of lux, CCT, and CL, and CL light optimization application 116 may be configured with one or more algorithms for determining an optimum or target value of CL.

Memory 110 may also include a calibration database 118 that may store calibration information specific to the particular lighting devices 102 being controlled by computing device 104. As described more below, calibration information can correlate lux, CCT, and CL values to current values for each of the two or more channels of solid state light sources, such as channels 202 or 302 (FIGS. 2A, 2B).

Computing device 104 may also include a communication module 120, in accordance with some embodiments. Communication module 120 may be configured, for example, to aid in communicatively coupling computing device 104 with: (1) lighting device 102; (2) light sensors 106, (3) UI 108, and/or (4) a network 122, if desired. Communication module 120 can be configured, for example, to execute any suitable wireless communication protocol that allows for data/information to be passed wirelessly. Each of computing device 104, lighting device 102, light sensors 106 and UI 108 can be associated with a unique ID (e.g., IP address, MAC address, cell number, or other such identifier) that can be used to assist the communicative coupling therebetween, in accordance with some embodiments. Some example suitable wireless communication methods that can be implemented by communication module 120 of computing device 104 may include: radio frequency (RF) communications (e.g., Zigee.®; Wi-Fi.®; Bluetooth.®; near field communication or NFC); IEEE 802.11 wireless local area network (WLAN) communications; infrared (IR) communications; cellular data service communications; satellite Internet access communications; custom/proprietary communication protocol; and/or a combination of any one or more thereof. In some embodiments, computing device 104 may be capable of utilizing multiple methods of wireless communication. In some such cases, the multiple wireless communication techniques may be permitted to overlap in function/operation, while in some other cases they may be exclusive of one another. In some cases a wired connection (e.g., USB, Ethernet, FireWire, or other suitable wired interfacing) may also or alternatively be provided between computing device 104 and the other components of lighting system 100.

In some instances, computing device 104 may be configured to be directly communicatively coupled with lighting device 102. In some other cases, however, computing device 104 and lighting device 102 optionally may be indirectly communicatively coupled with one another, for example, by an intervening or otherwise intermediate network 122 for facilitating the transfer of data between the computing device and lighting device. Network 122 may be any suitable communications network, and in some example cases may be a public and/or private network, such as a private local area network (LAN) operatively coupled to a wide area network (WAN) such as the Internet. In some instances, network 122 may include a wireless local area network (WLAN) (e.g., Wi-Fi® wireless data communication technologies). In some instances, network 122 may include Bluetooth® wireless data communication technologies. In some cases, network 122 may include supporting infrastructure and/or functionalities such as a server and a service provider, but such features are not necessary to carry out communication via network 122.

FIG. 2A is a functional block diagram of an example lighting device 102, which includes a plurality of differently-colored and independently-controllable channels 202_1 202_n, each channel including a plurality of modules 204, with each module including one or more solid state light source(s). A given solid-state emitter may be any semiconductor light source device, such as, for example, a light-emitting diode (LED), an organic light-emitting diode (OLED), a polymer light-emitting diode (PLED), or a combination thereof, among others. A given solid-state emitter may be configured to emit electromagnetic radiation (e.g., light), for example, from the visible spectral band, the infrared (IR) spectral band, the ultraviolet (UV) spectral band, or a combination thereof, among others.

As noted above, lighting system 100 may be configured to simultaneously control the CL, CCT, and lux of light generated by lighting device 102. In the illustrated example, adjustability is achieved via two or more differently-colored channels 202 of solid state light sources. In one example, each channel 202 may emit a substantially constant color of light that collectively define the extents of a controllable color space of light. In the illustrated example, at least one of channels 202 may emit a blue light having a peak wavelength of approximately 420 nm to approximately 480 nm and one or more channels may individually or collectively emit a white light. The one or more channels may be configured to emit white light having one or more CCTs. In one example, the one or more channels 202 may be configured to emit white light with a CCT of approximately 1800K-3000K, 2000K-3000K, 2000K-3500K, or 1800K-2700K, 3500K-6500K, 4000K-6500K, or 3000K-5000K, etc. In some examples, one or more of channels 202 may emit a color other than white or blue light, for example, one or more of red, green, yellow, and orange light.

Any construction technique known in the art for arranging the channels 202 of modules 204 may be used. In one example, modules 204 may be arranged on a printed circuit board in a checkerboard fashion and placed in an enclosure (not illustrated) with a diffuser (not illustrated) over the modules such that light emitted from the channels 202 of modules mix and present a substantially uniform color and intensity appearance to an observer. Modules 204 may be arranged in subgroupings or pixels of equal or differing numbers of modules from each channel. As would be understood by a person having skill in the art, other light components known in the art may be used, such as lenses, depending on the application of the particular lighting device 102. Lighting device 102 may also include additional components 206, which may include, by way of example, connectors, reverse polarity protection diodes, power supplies, and a printed circuit board. Computing device 104 (FIG. 1) is configured to adjust a relative output of channels 202 by adjusting a driving voltage or current to each of the channels to thereby simultaneously control the CCT, lux, and CL of the combined light output from lighting device 102.

As noted above, computing device 104 can be configured to independently control the intensity of each of channels 202 to thereby simultaneously control the CCT, lux, and CL of the light emitted by lighting device 102. CL light optimization application 116 (FIG. 1) may incorporate a mapping of light output for each channel 202 to cumulative lighting characteristics as described in equation 1:

$$( \begin{matrix}{I}_{1} \\ {I}_{2} \\ ⋮ \\ {I}_{n}\end{matrix} ) ⇒ ( \begin{matrix}lux \\ CCT \\ CL\end{matrix} ) \qquad Eq . ( 1 )$$

in which:

I₁₋ₙ is the intensity of the light output from each of channels 202_1-n, respectively; and

lux, CCT, and CL are the lux, CCT, and CL of the cumulative light output of all channels 202.

Thus, CL light optimization application 116 may incorporate a mapping from an n-dimensional space of intensities (n=number of channels 202) to a 3-dimensional space of values of lux, CCT, and circadian light of the sum of all of the channels. In one example, a method of controlling lighting device 102 may include specifying the values on the right side of Equation 1, for example, based on input provided by one or both of light sensors 106 and UI 108, and determining the corresponding values for the intensity of each channel 202 (left side of Equation 1) to achieve the specified target lighting values. Because not all values of lux, CCT, and CL can be achieved simultaneously, CL light optimization application 116 may also include information on the universe of allowed simultaneous values of lux, CCT, and CL.

Example Implementation

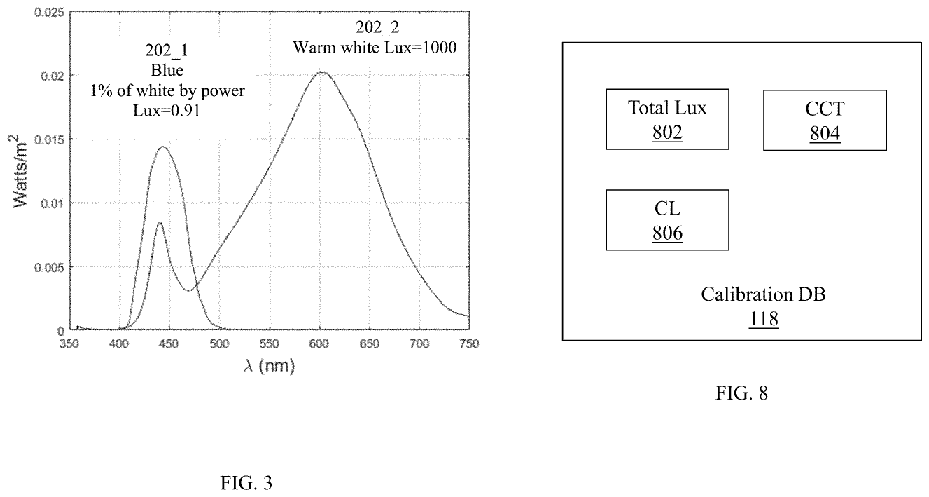

FIG. 2B illustrates one example implementation of lighting device 300 that may be used for lighting device 102 (FIG. 1) that includes a blue channel 302_1 and a warm white channel 302_2. FIG. 3 is a graph of spectral emission curves for example lighting device 300, plotted at arbitrarily chosen intensities. As discussed more below, such a combination of color channels (e.g. channels 202, 302) may be used, for example, to increase or decrease the Circadian Light (e.g., increase the intensity of blue channel 302_1) while not causing an appreciable change in CCT or lux of the cumulative light output because the cumulative CCT and lux is established mainly by white channel 302_2.

In the illustrated example, the vertical scale in FIG. 3 is calibrated such that white channel 302_2 produces 1000 Lux (lumens/m2). For illustrative purposes, blue channel 302_1 intensity is 1% of white channel 302_2 by power, for which the blue channel produces 0.91 lux. Thus, in the illustrated example, if the intensity of blue channel 302_1 is the same as white channel 302_2, it would have a lux of 91. The relationship of blue channel 302_1 lux and intensity to white channel 302_2 lux and intensity in this example can thus be described as follows:

blue lux=0.091×white lux×fractional blue intensity Eq. (2)

total lux=(1+0.091×fractional blue intensity)×white lux Eq. (3)

Thus, blue channel 302_1 contributes very little to the total lux (9.1% if the blue channel intensity were the same as white channel 302_2 intensity).

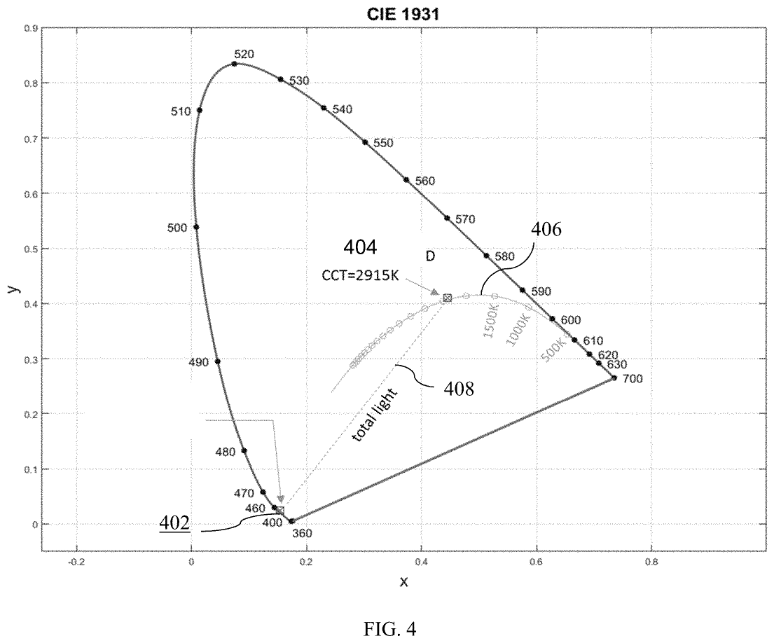

FIG. 4 is a CIE chromaticity diagram for example blue channel 302_1 and white channel 302_2, with a location 402 of the blue channel and a location 404 of the white channel light emitters shown. Numbers around the periphery of the diagram are wavelengths in nm. The circles along the blackbody locus 406 are temperature markers every 500K starting at 500K. In the illustrated example, warm white channel 302_2 has a CCT of 2915K. Blue channel 302_1 has an indeterminate CCT because it does not lie along an isothermal temperature line that intersects blackbody locus 406.

The color of the total light output by lighting device 102 with the illustrated example blue and warm white channels 302_1, 302_2 can be varied along line 408 joining the blue channel and white locations 402, 404 by varying the intensities of the blue channel and the white channel. The ratio of the distance to blue channel location 402 to the distance to white channel location 404 at any operating point along line 408 is equal to a ratio of white channel 302_2 intensity to blue channel 302_1 intensity.

CL light optimization application 116 may be configured with a mapping of the intensity of blue channel 302_1 and white channel 302_2 to total light output according to Equation 1 above and may be configured to determine a driving current or voltage for each of the channels to obtain a target intensity to obtain a target CCT, lux, and CL.

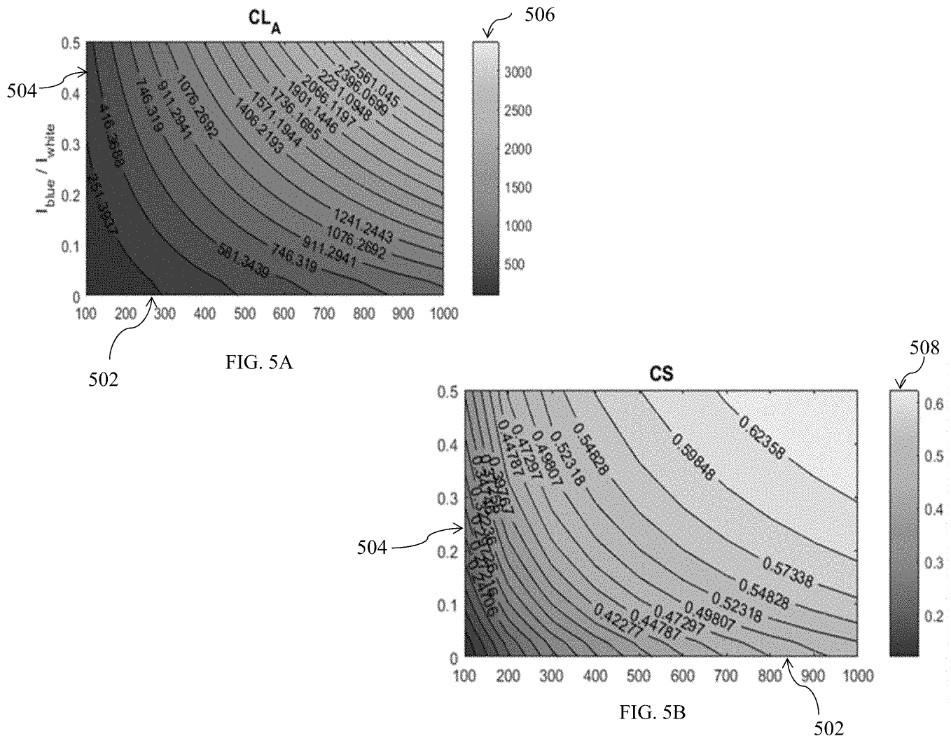

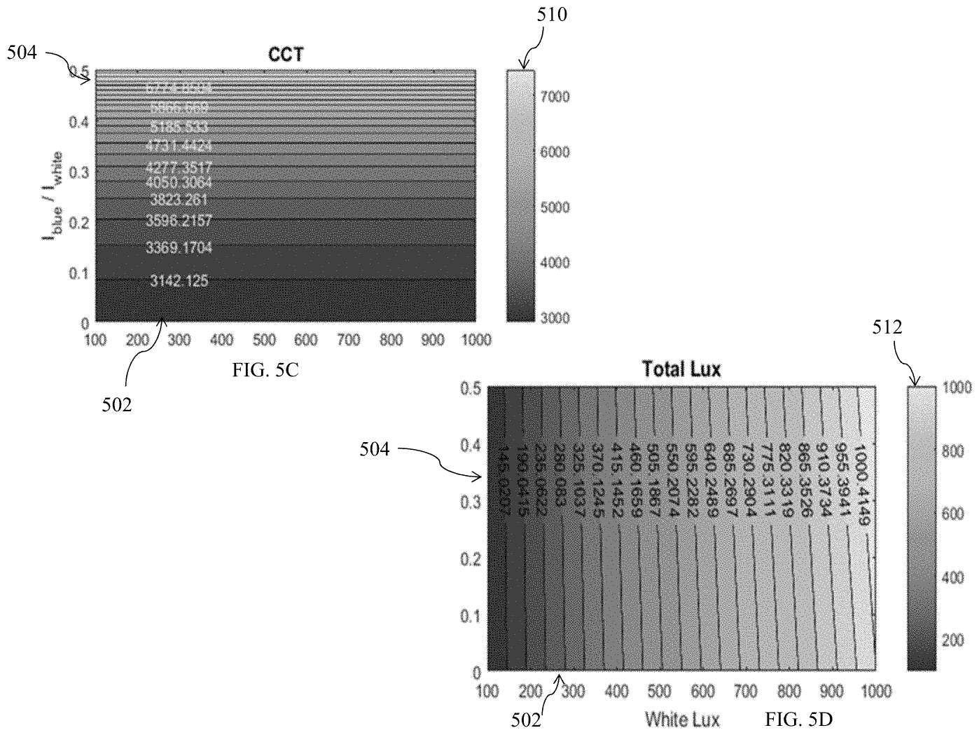

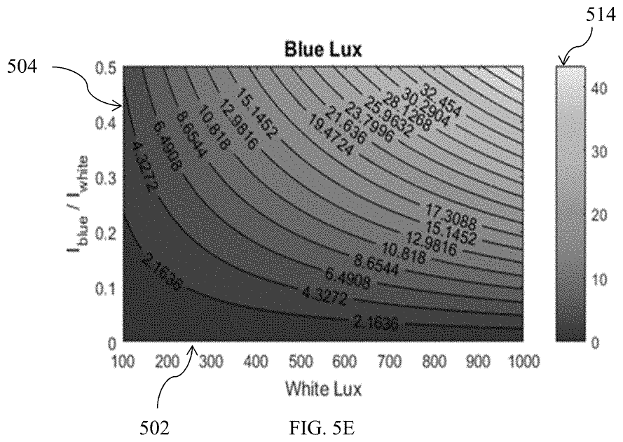

FIGS. 5A-5E show contours of various quantities of interest that may be utilized by computing device 104 to control lighting device 102. In the illustrated example, each of FIGS. 5A-5E show contour lines of a quantity of interest as a function of the lux of white channel 302_2 along the x-axis 502 and the ratio of the intensity of blue channel 302_1 to white channel 302_2 (referred to herein as fractional blue intensity and in the accompanying figures as I_{blue}/I_{white}) along the y-axis 504. The contours in FIGS. 5A-5E use white lux rather than white intensity as the independent variable (x-axis 502) because the lux of white channel 302_2 is proportional to the intensity of the white channel and because lux generally has a more intuitive meaning than intensity for human vision applications. Similar contours, however, may be generated with intensity of white channel 302_2 or blue channel 302_1 as the independent variable.

Within each FIG. 5A-5E, changes in level of adjacent contours are all the same. FIGS. 5A and 5B illustrate two particular and related CL metrics, CL_{A} 506 and CS 508. As described more below, there is currently no metric for circadian light that is recognized as a standard by any of the officially recognized and/or legal standards organizations. However, there is wide body of work from the past ˜40 years from which a substantial understanding has developed about the interaction of light with the HCS. For purposes of illustration, the present disclosure applies a modified version of a CL metric developed at the Lighting Research Center (LRC) at Rensselaer Polytechnic Institute (RPI) in Troy, N.Y. Mathematical details of the metric are provided below. As will be appreciated by a person having ordinary skill in the art, any CL metric currently in existence or developed at a later date could be applied with the systems and methods of the present disclosure.

FIGS. 5A and 5B show the contours of two related LRC metrics—CL_{A} 506 and CS 508—in which CL_{A} is a measure of the level of circadian light, or the light's ability to suppress the production of melatonin by the pineal gland in the hypothalamus, and CS is a simple rescaling of CL_{A} to provide a metric that is roughly proportional to the percent melatonin suppression under given lighting conditions, where a CS of 0 indicates no melatonin suppression and the maximum possible value of CS is 0.7, which indicates an approximate 100% suppression of melatonin production. FIG. 5C is a contour plot of CCT 510 of light emitted by lighting device 102, 5D is a contour plot of total lux 512 and FIG. 5E a contour plot of blue channel 302_1 lux 514.

As can be seen in FIGS. 5A and B, circadian light (CL_{A} 506 and CS 508) is increased by increasing either white lux 502 or fractional blue intensity 504. However, in FIG. 5C, CCT 510 is influenced almost entirely by fractional blue intensity 504. CCT 510 changes at first slowly with increasing fractional blue intensity 504, and then more quickly. This is opposite for Circadian Light (CL_{A} 506 and CS 508), where more substantial increases in CL_{A} and CS occur with small changes in fractional blue intensity 504. FIGS. 5D-E reflects what was discussed above in connection with Equations (2) and (3)—that total lux 512 is almost entirely influenced by the intensity or lux of white channel 302_2. Thus, FIGS. 5A-E show that blue channel 302_1 can be used as a “knob” to control a level of CL over certain ranges without having an appreciable impact on CCT or total lux, for which blue channel 302_1 has less influence.

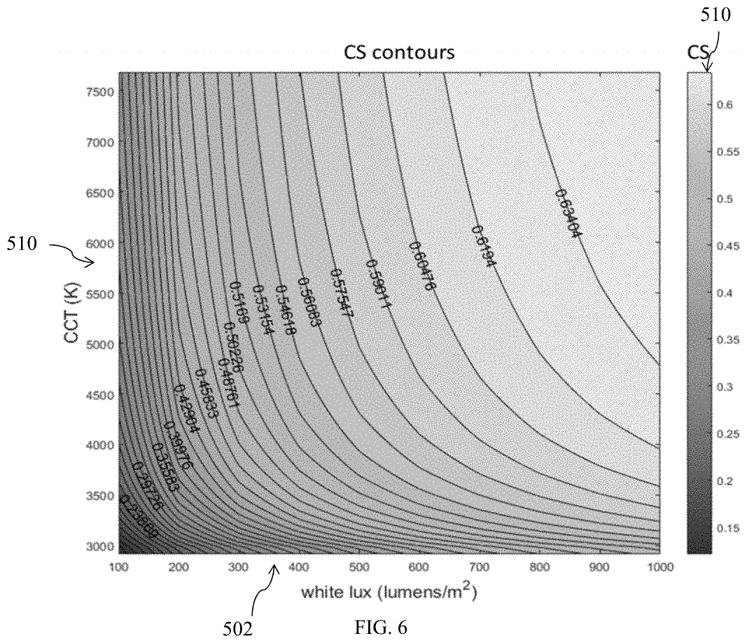

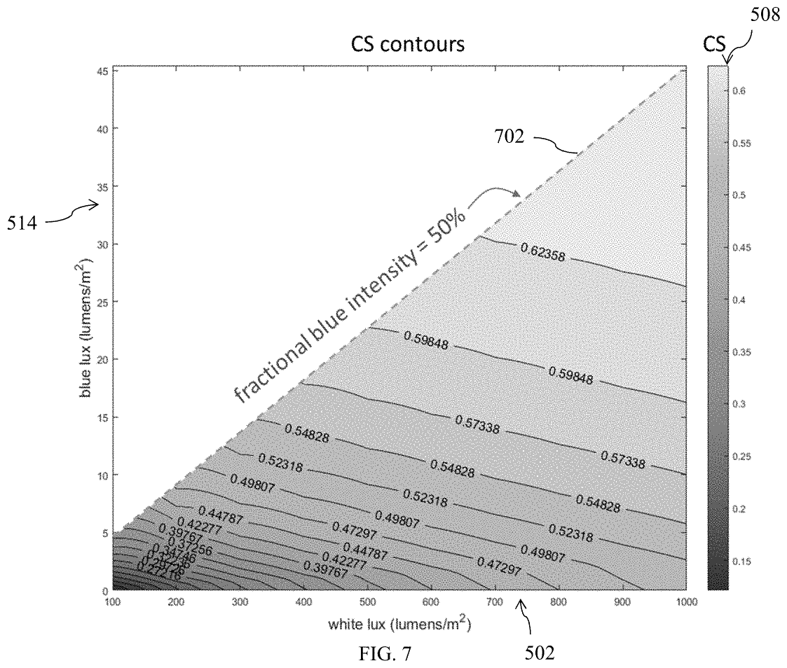

FIGS. 6 and 7 demonstrate other ways of expressing the relationship between the intensity of channels 302_1 and 302_2 and the characteristics of the light emitted by the two channels. FIG. 6 shows contours of CS 508 with respect to white light lux 502 and CCT 510. By going vertically up the graph, one increases CCT 510, fractional blue intensity 504, and fractional blue lux, while maintaining a constant white lux 502 (and total lux 512) nearly the same. The tight grouping of CS contour lines 508 in the left and lower-left portions of FIG. 6 also illustrate how larger changes in CL (CS 508) can be achieved with small changes in total lux and CCT, particularly at lower lux and warmer lighting conditions. FIG. 7 shows contours of CS 508 with respect to white lux 502 and blue lux 514. The maximal fractional blue intensity considered in the illustrated example is 50% (which occurs along dashed line 702). Lines parallel to dashed line 702 correspond to constant fractional blue intensity either greater than 50% (above the dashed line) or less than 50% (below the dashed line). The CS contour plot shown in FIG. 7 and the associated data used to generate the plot could be utilized by a lighting controller, such as computing device 104 (FIG. 1), to determine the intensity or lux levels for each of channels 202 (FIG. 2) to achieve a desired CL. FIGS. 5-7 illustrate the correlation between lux, CCT, and CL. With knowledge of such correlations, computing device 104 can be configured to receive or determine a target or range for one or more of lux, CCT, and CL and then determine a range of achievable values for other ones of the lux, CCT, and CL. For example, for a given target value or range of values for each of lux and CCT, the computing device 104 can determine a range of achievable CL values. For example, computing device 104 can determine the extent to which the intensity of blue channel 302_1 may be varied to maximize or minimize CL for purposes of LRH while still providing a combined light output from the lighting device 102 that has a CCT and lux within the target range.

Table 1 provides example total lux levels that may be recommended or desirable for particular activities. Table 1 was obtained from Engineering ToolBox, Illuminance—Recommended Light Level (2004) (available at: https://www.engineeringtoolbox.com/light-level-rooms-d_708.html). In one example, memory 110 may include a table of total lux levels for particular activities or settings and may also include user-defined lux levels, for example, for particular lighting modes, such as reading, dinner, TV, computer work, etc. Memory 110 may similarly include tables of CCT and CL values for various activities or lighting modes that can be used as target control values for controlling lighting device 102.

TABLE 1

| | Total Lux |
|---|---|
| Activity | (lumen/m\hat{ }2) |
| Public areas with dark surroundings | 20-50 |
| Simple orientation for short visits | 50-100 |
| Working areas where visual tasks are only occasionally | 100-150 |
| performed | |
| Warehouses, Homes, Theaters, Archives | 150 |
| Easy Office Work, Classes | 250 |
| Normal Office Work, PC Work, Study Library, Groceries, | 500 |
| Show Rooms, Laboratories | |
| Supermarkets, Mechanical Workshops, Office Landscapes | 750 |
| Normal Drawing Work, Detailed Mechanical Workshops, | 1,000 |
| Operation Theaters | |
| Detailed Drawing Work, Very Detailed Mechanical Works | 1500-2000 |
| Performance of visual tasks of low contrast and very | 2000-5000 |
| small size for prolonged periods of time | |
| Performance of very prolonged and exacting visual tasks | 5000-10000 |
| Performance of very special visual tasks of extremely | 10000-20000 |
| low contrast and small size | |

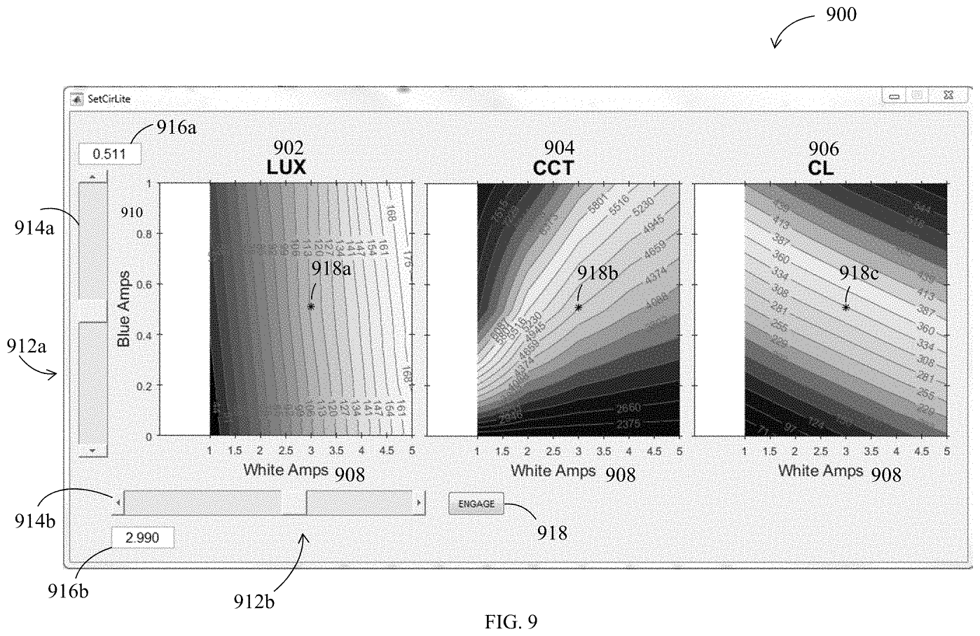

FIG. 8 shows one example of tables that may be included in calibration database 118 (see also FIG. 1). In the illustrated example, calibration database 118 may include a total lux table 802, a CCT table 804, and a CL table 806 that define the lux, CCT, and CL, respectively, for a range of channel values, such as driving current, for each of channels 202 or 302. Calibration database may, therefore, provide lighting information that includes the lux, CCT, and CL of light output by lighting device 102 as a function of an output of a driving current or voltage for two or more channels of light sources, such as channels 202 or 302. FIG. 9 illustrates an example user interface (UI) 900 that may be incorporated into UI 108 (FIG. 1). UI 900 includes a total lux contour plot 902 of the data in total lux table 802, a CCT contour plot 904 of the data in CCT table 804, and a CL contour plot 906 of the data in CL table 806. In the illustrated example, each contour plot is plotted versus white channel 302_2 driving current 908 and blue channel 302_1 driving current 910. The illustrated UI 900 includes blue and white channel control panels 912a and 912b, which each include a slide 914a, 914b for setting a driving current level for channels 202_1, 202_2, a digital display 916a, 916b for numerical input and display of the channel current level in, e.g., amps, and an engage button 918 for implementing the combination of driving currents 908, 910 thus selected for driving lighting device 102 (in one example, rather than selecting engage button 918, the driving currents may be automatically engaged upon changing slides 914a and 914b). UI 108 also includes operating point indicators 918a, 918b, and 918c as shown in FIG. 9 that indicate the total lux, CCT, and CL of the light output by lighting device 102 for a particular combination of driving currents to the channels (e.g., 202, 302) of the lighting device. A user may, therefore use control panels 912a, 912b to vary the driving current to each channel 202 and observe the resulting change in the characteristics of light emitted by lighting device 102.

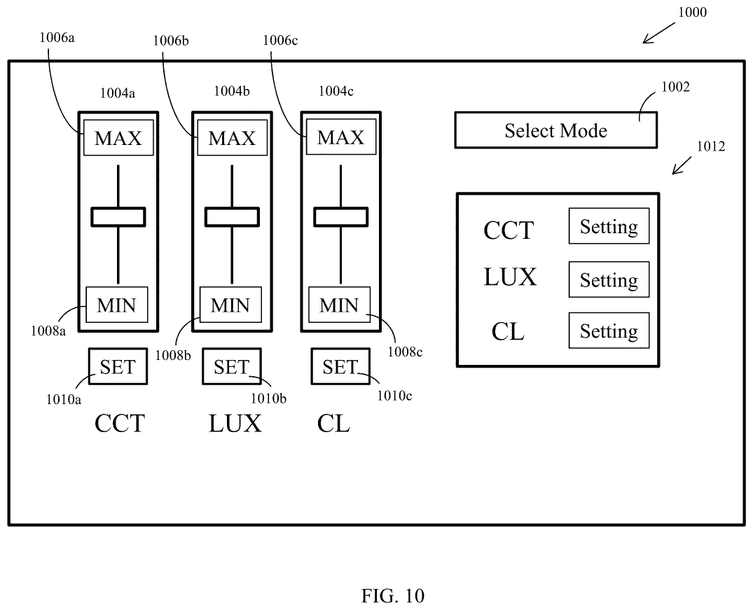

FIG. 10 illustrates another example UI 1000 that may be incorporated in UI 108 (FIG. 1). UI 1000 includes a user-selectable lighting mode 1002, which may be implemented in a variety of ways, such as a drop down menu, for selecting a lighting mode for an activity, such as one of the activities listed above in Table 1, which may include predefined lux, CCT, and CL values. UI 1000 may also include control features 1004a-c for independent user control of CCT, lux, and CL, respectively. In the illustrated example, control features 1004 are dimmer slides, implemented via a graphical display or physical dimmer slides. Control features 1004 also include a digital display of the maximum 1006a-c and minimum 1008 a-c available CCT, lux, and CL, and set buttons 1010a, 1010b, 1010c, for setting one or more of CCT, lux, and CL at a specific value. As discussed above, CCT, lux, and CL are not fully independent values. As a user modifies the desired level of one of CCT, lux, and CL, UI 1000 may dynamically update the available range of settings for the other ones of CCT, lux, and CL by updating the maximum 1006 values and minimum 1008 values. UI 1000 may also include a display 1012 for displaying the current CCT, lux, and CL of light being emitted by lighting device 102. UI 1000 may, therefore, be used to specify one or more of CCT, lux, and CL (for example, the right hand side of Equation 1) and CL light optimization application 116 may be configured to determine a corresponding driving current for each of channels 202 or 302 to achieve the user-specified combination of CCT, lux, and CL. Thus, a user may utilize UI 1000 to select a lux and CCT that he or she finds visually appealing or appropriate for an activity, and then adjust CL control feature 1004 to adjust the CL of the light output for improved LRH. As discussed above, depending on the lighting values, the user may be able to adjust the CL of the light for improved LRH without changing the light characteristics in a way that is noticeable to the user. As noted above, a wearable device may also be used to automatically specify the CL of lighting device 102. And in other examples, computing device 104 may be configured to automatically set one or more of CCT, lux, and CL.

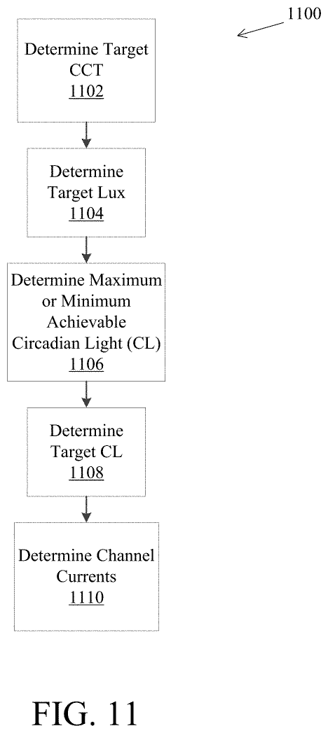

FIG. 11 is a flowchart of an example method 1100 of controlling a lighting device, such as lighting device 102 for LRH. Method 1100 may be performed by a computing device (e.g., computing device 104) operatively connected to one or more lighting devices. In block 1102, the computing device may determine a target CCT range or target CCT value for light output by a lighting device, such as lighting device 102. For example, computing device 104 may include instructions for determining a desired CCT based on, for example, a user-specified lighting mode, a time of day, or based on input from one or more sensors, such as light sensors 106. Computing device 104 may also receive a target CCT or range of CCT values, for example, from a UI, such as UI 108. In block 1104, the computing device may similarly determine a target lux range or receive a target lux range or value. At block 1106, the computing device may determine a maximum or minimum achievable CL value based on the target CCT and Lux ranges or values. For example as noted above, the CCT, lux, and CL of a light source are not fully independent values. The computing device may include a CL light optimization application, such as CL light optimization application 116 for determining a range of available CL values for a given universe of CCT and lux values. At block 1108, after determining a range of available CL values, the computing device may determine a target CL value. For example, the computing device may receive a CL instruction from a wearable device that instructs the lighting device to emit light having a specific CL, or within a range of CLs, or to minimize or maximize CL within the range of achievable values for a given target CCT and lux. In other examples, CL light optimization application may be configured to determine a desired CL based on one or more inputs, such as time of day, day of the year, lighting mode, etc. As will be appreciated, the order of blocks 1102 to 1108 may be varied. For example, instead of first specifying CCT and lux and then determining CL, any order of specifying one or more of CCT, lux, and CL and determining the available range of the other parameter(s) may be used. At block 1110, the computing device may determine the driving currents for each lighting channel of the lighting device, e.g., channels 202 or 302, to achieve the target CCT, lux, and CL values, using, for example, calibration data, e.g., calibration data stored in calibration database 118. The computing device can then send one or more instructions to the lighting device to cause the lighting device to output a light with the target CCT, lux, and CL values.

Example Calculation of a CL Metric

As noted above, at present, there is no metric that is recognized as a standard by any of the officially recognized and/or legal standards organizations. However, there is a wide body of work from the past ˜40 years from which a substantial understanding has developed about the interaction of light with the HCS. From these, certain metrics have been developed that provide good indication of the effectiveness of the light from HCS viewpoint. Similar to lux and CCT, these metrics are based on the spectrum of the light and, more specifically, are generally based on the conclusion that the HCS is most sensitive to the blue end of the visible spectrum.

For the purposes of discussion and demonstration, the present application utilizes a particular metric with some small modifications based on one developed at the Lighting Research Center (LRC) at Renselaer Polytechnic Institute (RPI) in Troy, N.Y. The LRC uses a metric known as “Circadian Light”, abbreviated CL_{A}. The LRC also introduces a related metric, Circadian Stimulus (CS), which is a rescaling of CL_{A} by a straightforward mathematical transformation, and therefore substantively the same metric. As noted above, the teachings of the present disclosure is easily adaptable to other CL metrics that may be more appropriate for a particular application, or that may become available as the science and understanding of circadian light matures.

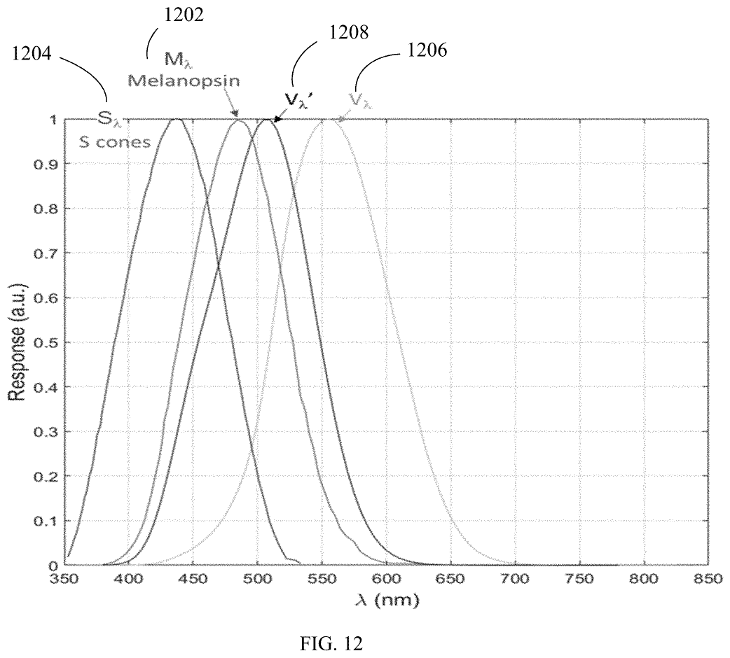

The quantity CL_{A} is an encapsulation of two aspects of the circadian system's response to light. The first is that its spectral response (in the blue part of the spectrum) is due to a combination of absorption by melanopsin (present in the retinal ganglia cells in the eye) and the S-cones (the blue sensitive cones in the retina). The second is that there is an “opponency” that comes into play in strong blue vision conditions between, on one side, the blue light sensing of the S cones and, on the other side, the green plus red light sensing of the M and L-cones, and the rod cell response. The former in combination are responsible for the lumen curve, V_{λ} (so-called photopic vision) and the latter is associated with night vision (scotopic vision) and typically denoted V_{λ}′. Thus, for CL_{A} there are four spectral responsivity curves to consider, which are plotted in FIG. 12, normalized by their own respective peaks: melanopsin (M_{λ}) 1202, S-cone (S_{λ}) 1204, photopic lumen curve (V_{λ}) 1206, and scotopic lumen curve (V_{λ}′) 1208.

If the spectral intensity of the light is I_{λ}, then each of the four channels senses the integral over wavelength of the respective spectral responsivity curve (FIG. 12) multiplied by the spectral intensity. If these are denoted V, V′, M, and S then CL_{A} is given as follows:

$${CL}_{A} = 1548 \times \{ \begin{matrix}[ M + {a}_{b - y} ( S - kV ) - {a}_{rod} ( 1 - exp [ - {V}^{′} / RodSat ] ) ] & if S - kV > 0 \\ M & if S - kV < 0\end{matrix} \qquad Eq . ( 4 )$$

in which

a_{b-y}=0.7

a_{rod}=3.3

RodSat=6.5

k=0.2616

The upper expression Equation (4) expresses the opponency mentioned above. The greater the value of CL_{A}, the greater the melatonin suppression. The circadian stimulus, CS, is a rescaling of CL_{A} in order to have a metric which is roughly proportional to the percent melatonin suppression under given lighting conditions. CS is given as follows:

$$CS = 0.7 - \frac{0.7}{1 + {( \frac{{CL}_{A}}{355.7} )}^{1.1026}} \qquad Eq . ( 5 )$$

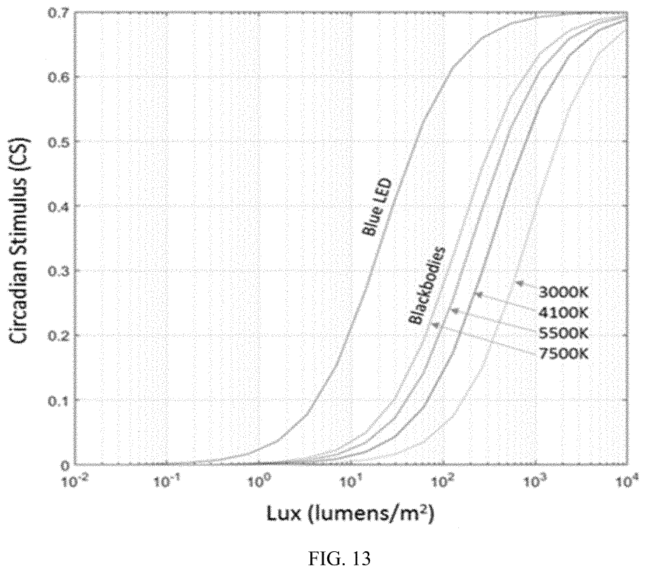

CS is in the range of 0 (CL_{A}=0, no melatonin suppression) to a saturation value of 0.7 (CL_{A}=∞, 100% melatonin suppression). FIG. 13 shows examples of CS for various light sources (blue LED and four blackbodies of different temperatures) over a range of Lux. For the blackbodies, the temperatures are also equal to their CCT values.

Any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a wearable device (e.g., smart watch), a web appliance, a network router, a network switch, a network bridge, any machine capable of executing a sequence of instructions that specify an action to be taken by that machine, and any combinations thereof. In one example, a computing device may include and/or be included in a kiosk.

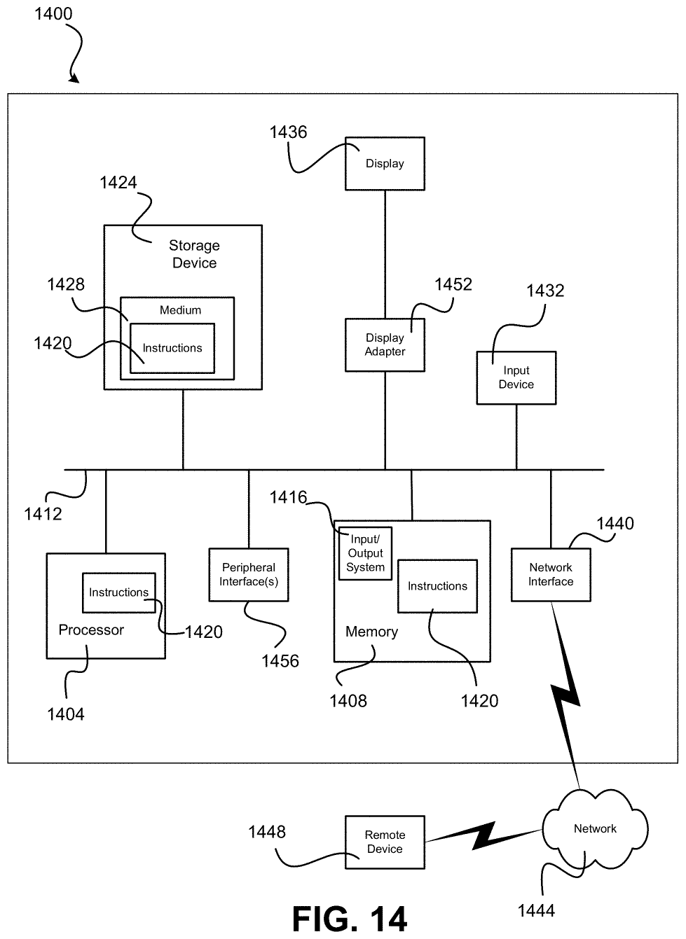

FIG. 14 shows a diagrammatic representation of one embodiment of a computing device in the exemplary form of a computer system 1400 within which a set of instructions for causing a control system, such as lighting system 100 of FIG. 1, to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1400 includes a processor 1404 and a memory 1408 that communicate with each other, and with other components, via a bus 1412. Bus 1412 may include any of several types of bus structures including, but not limited to, a memory bus, a memory controller, a peripheral bus, a local bus, and any combinations thereof, using any of a variety of bus architectures.

Memory 1408 may include various components (e.g., machine-readable media) including, but not limited to, a random access memory component, a read only component, and any combinations thereof. In one example, a basic input/output system 1416 (BIOS), including basic routines that help to transfer information between elements within computer system 1400, such as during start-up, may be stored in memory 1408. Memory 1408 may also include (e.g., stored on one or more machine-readable media) instructions (e.g., software) 1420 embodying any one or more of the aspects and/or methodologies of the present disclosure. In another example, memory 1408 may further include any number of program modules including, but not limited to, an operating system, one or more application programs, other program modules, program data, and any combinations thereof.

Computer system 1400 may also include a storage device 1424. Examples of a storage device (e.g., storage device 1424) include, but are not limited to, a hard disk drive, a magnetic disk drive, an optical disc drive in combination with an optical medium, a solid-state memory device, and any combinations thereof. Storage device 1424 may be connected to bus 1412 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1424 (or one or more components thereof) may be removably interfaced with computer system 1400 (e.g., via an external port connector (not shown)). Particularly, storage device 1424 and an associated machine-readable medium 1428 may provide nonvolatile and/or volatile storage of machine-readable instructions, data structures, program modules, and/or other data for computer system 1400. In one example, software 1420 may reside, completely or partially, within machine-readable medium 1428. In another example, software 1420 may reside, completely or partially, within processor 1404.

Computer system 1400 may also include an input device 1432. In one example, a user of computer system 1400 may enter commands and/or other information into computer system 1400 via input device 1432. Examples of an input device 1432 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1432 may be interfaced to bus 1412 via any of a variety of interfaces (not shown) including, but not limited to, a serial interface, a parallel interface, a game port, a USB interface, a FIREWIRE interface, a direct interface to bus 1412, and any combinations thereof. Input device 1432 may include a touch screen interface that may be a part of or separate from display 1436, discussed further below. Input device 1432 may be utilized as a user selection device for selecting one or more graphical representations in a graphical interface as described above.

A user may also input commands and/or other information to computer system 1400 via storage device 1424 (e.g., a removable disk drive, a flash drive, etc.) and/or network interface device 1440. A network interface device, such as network interface device 1440, may be utilized for connecting computer system 1400 to one or more of a variety of networks, such as network 1444, and one or more remote devices 1448 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1444, may employ a wired and/or a wireless mode of communication. In general, any network topology may be used. Information (e.g., data, software 1420, etc.) may be communicated to and/or from computer system 1400 via network interface device 1440.

Computer system 1400 may further include a video display adapter 1452 for communicating a displayable image to a display device, such as display device 1436. Examples of a display device include, but are not limited to, a liquid crystal display (LCD), a cathode ray tube (CRT), a plasma display, a light emitting diode (LED) display, and any combinations thereof. Display adapter 1452 and display device 1436 may be utilized in combination with processor 1404 to provide graphical representations of aspects of the present disclosure. In addition to a display device, computer system 1400 may include one or more other peripheral output devices including, but not limited to, an audio speaker, a printer, and any combinations thereof. Such peripheral output devices may be connected to bus 1412 via a peripheral interface 1456. Examples of a peripheral interface include, but are not limited to, a serial port, a USB connection, a FIREWIRE connection, a parallel connection, and any combinations thereof.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure.

Exemplary embodiments have been disclosed above and illustrated in the accompanying drawings. It will be understood by those skilled in the art that various changes, omissions and additions may be made to that which is specifically disclosed herein without departing from the spirit and scope of the present disclosure.

What is claimed is:

1. A method of controlling a lighting device having at least two differently colored channels of solid state light sources, comprising:
independently controlling an output of the at least two channels to simultaneously control a lux, correlated color temperature (CCT), and circadian light (CL) of light emitted by the lighting device.

2. The method of claim 1, wherein the at least two channels include a blue channel including a plurality of blue solid state light sources and a white channel including a plurality of white solid state light sources, the method further comprising controlling the CL of the light emitted by the lighting device predominately with the blue channel and controlling the lux of the light emitted by the lighting device predominately with the white channel.

3. The method of claim 1, wherein the at least two channels include a first white channel including a plurality of white solid state light sources having a first CCT and a second white channel including a plurality of white solid state light sources having a second CCT, the first and second CCTs being different.

4. The method of claim 3, wherein the at least two channels further include a blue channel including a plurality of blue solid state light sources.

5. The method of claim 1, further comprising:
receiving target values for the lux, CCT, and CL of light emitted by the lighting device;
receiving lighting information that includes the lux, CCT, and CL of light output by the lighting device as a function of a driving current or voltage for each of the at least two channels; and
determining, with the lighting information, a driving current or voltage for each of the at least two channels to emit light from the lighting device having the target values of lux, CCT, and CL.

6. The method of claim 1, further comprising:
receiving a calibration database that includes a lux table, a CCT table, and a CL table that define the lux, CCT, and CL, respectively, of light output by the lighting device as a function of driving current or voltage for the at least two channels; and
determining an output for each of the at least two channels according to the calibration database.

7. The method of claim 1, further comprising:
receiving a target lux and CCT;
determining a range of CL values that is achievable at the target lux and CCT;
determining whether CL should be minimized or maximized;
determining a target CL within the range of achievable CL values; and
determining an output of the at least two channels to emit a combined light having the target lux, CCT, and CL.

8. The method of claim 1, further comprising:
receiving at target value for at least one of the lux, CCT, and CL of light output by the lighting device;
determining a range of achievable values for the other ones of the lux, CCT, and CL of the light output;
determining a target value within the range of achievable values for each of the other ones of lux, CCT, and CL; and
determining a driving current or voltage for each of the at least two channels to emit a combined light output having the target lux, CCT, and CL.

9. The method of claim 1, further comprising:
receiving a target lux and CCT;
receiving a CL instruction;
determining a target CL of the light output by the lighting device according to the target lux and CCT and the CL instruction;
determining a driving current for each of the at least two channels to emit a combined light having the target lux, CCT, and CL; and
sending a control signal to the lighting device to emit light having the target lux, CCT, and CL.

10. The method of claim 1, further comprising:
providing a user interface (UI) that includes control features for simultaneously controlling the CL and lux of the light output by the lighting device.

11. The method of claim 1, further comprising:
providing a user interface (UI) that includes control features for simultaneously controlling the CL, lux, and CCT of the light output by the lighting device.

12. A lighting system, comprising:
a lighting device including at least two differently colored channels of solid state light sources; and
a processor coupled to the lighting device and configured to independently control an output of the at least two channels to simultaneously control a lux, correlated color temperature (CCT), and circadian light (CL) of light emitted by the lighting device.

13. The lighting system of claim 11, wherein the at least two channels include a blue channel including a plurality of blue solid state light sources and a white channel including a plurality of white solid state light sources, wherein the processor is further configured to:
control the CL of the light emitted by the lighting device predominately with the blue channel and control the lux of the light emitted by the lighting device predominately with the white channel.

14. The lighting system of claim 11, wherein the at least two channels include a first white channel including a plurality of white solid state light sources having a first CCT and a second white channel including a plurality of white solid state light sources having a second CCT, the first and second CCTs being different.

15. The lighting system of claim 11, further comprising a non-transitory computer readable medium containing lighting information that includes the lux, CCT, and CL of light output by the lighting device as a function of a driving current or voltage for each of the at least two channels, wherein the processor is further configured to:
receive target values for the lux, CCT, and CL of light emitted by the lighting device; and
determine, with the lighting information, a driving current or voltage or each of the at least two channels to emit light with the lighting device having the target values of lux, CCT, and CL.

16. The lighting system of claim 11, further comprising a non-transitory computer readable medium containing a calibration database that includes a lux table, a CCT table, and a CL table that define the lux, CCT, and CL, respectively, of light output by the lighting device as a function of a driving current or voltage for the at least two channels, wherein the processor is further configured to:
determine an output for each of the at least two channels according to the calibration database.

17. The lighting system of claim 11, wherein the processor is further configured to:
receive a target lux and CCT;
determine a range of CL values that is achievable at the target lux and CCT;
determine whether CL should be minimized or maximized;
determine a target CL within the range of achievable CL values; and
determine an output of the at least two channels to emit a combined light having the target lux, CCT, and CL.

18. The lighting system of claim 11, wherein the processor is further configured to:
receive at target value for at least one of the lux, CCT, and CL of light output by the lighting device;
determine a range of achievable values for the other ones of the lux, CCT, and CL of the light output;
determine a target value within the range of achievable values for each of the other ones of lux, CCT, and CL; and
determine a driving current or voltage for each of the at least two channels to emit a combined light output having the target lux, CCT, and CL.

19. The lighting system of claim 11, wherein the processor is further configured to:
receive a target lux and CCT;
receiving a CL instruction;
determine a target CL of the light output by the lighting device according to the target lux and CCT and the CL instruction;
determine a driving current for each of the at least two channels to emit a combined light having the target lux, CCT, and target CL; and
send a control signal to the lighting device to emit light having the target lux, CCT, and CL.

20. The lighting system of claim 11, wherein the processor is further configured to:
provide a user interface (UI) that includes control features for simultaneously controlling the CL, lux, and CCT of the light output by the lighting device.
